(19) United States
(12) Patent Application Publication
(10) Pub. No.: US 2011/0262959 A1
(43) Pub. Date: Oct. 27, 2011

(54) METHOD FOR EVALUATION OF DIFFERENTIATION ABILITY OF STEM CELL

(75) Inventors: Eiji Kobayashi, Wakayama (JP); Takashi Yokoo, Tochigi (JP); Yutaka Hanazono, Tochigi (JP); Hiroshi Nagashima, Kanagawa (JP)

(73) Assignees: JICHI MEDICAL UNIVERSITY, Shimotsuke-shi (JP); OTSUKA PHARMACEUTICAL FACTORY, INC., Naruto-shi (JP)

(21) Appl. No.: 13/142,222

(22) PCT Filed: Jun. 30, 2009
(86) PCT No.: PCT/JP2009/061981

§ 371 Date: Jul. 15, 2011

(30) Foreign Application Priority Data
Dec. 26, 2008 (JP) PCT/JP2008/073849

(51) Int. Cl. C12Q 1/02

(52) U.S. Cl. 435/29

(57) ABSTRACT

The present invention provides a method of evaluating the possibility of a stem cell being able to differentiate into a cell that constitutes a desired tissue in a living organism, comprising the following steps of:
(1) transplanting a stem cell to be evaluated into a primordium of a desired tissue of a non-human mammal,
(2) culturing the tissue primordium in vitro, and
(3) determining the possibility that the stem cell differentiates into a cell that constitutes the tissue in a living organism, with the degree of the dispersion of cells derived from the transplanted stem cell in the cultured tissue primordium as an index.

### TECHNICAL FIELD

The present invention relates to a method of determining whether a stem cell is capable of differentiating into a cell that constitutes a desired tissue in a living organism.

### BACKGROUND ART

Recent years' advances in stem cell research have led to the establishment of a wide variety of stem cells that exhibit pluripotency. To appropriately regenerate organs and tissues using these stem cells at the clinical level, it is important to confirm in advance that the stem cells to be used are capable of appropriately differentiating into the desired organ and constructing the normal tissue morphology.

Of the stem cells that have been established to date, ES cells and iPS cells proliferate very efficiently and have the capability of differentiating into a wide variety of cells (Non-patent Document 1), but when transplanted to a living organism as they are, they often fail to contribute to the construction of normal tissue and form a teratoma (Non-patent Document 2). Meanwhile, somatic stem cells such as mesenchymal stem cells (MSCs) often undergo replicative senescence in passage culture in vitro, and when transplanted to a living organism, they differentiate into a wide variety of cells without tumorigenesis and contribute to the construction of normal tissue (Non-patent Document 3). However, to find which of the above-described two properties the stem cell possesses, many repeated cultivations and differentiation induction tests must be performed, which consumes a great deal of time. Accordingly, there is a demand for the establishment of a method of conveniently screening for which of the above-described properties various stem cells possess, and whether the stem cells have been improved to prevent teratogenesis, so as to speedily develop stem cells that accurately differentiate into a tissue or organ.
non-patent document 1: Nagata M. et al., J. Gene Med., vol. 5; p. 921, 2003
non-patent document 2: Asano T. et al., Methods Mol. Bio., vol. 329; p. 459, 2006
non-patent document 3: Hara M. et al., J. Autoimmun., vol. 30; p. 163, 2008

### SUMMARY OF THE INVENTION

### Problems to be Solved by the Invention

It is an object of the present invention to provide a method of conveniently determine whether a stem cell is a cell that forms a tumor such as a teratoma in a living organism or a cell capable of differentiating into a wide variety of cells without tumorigenesis in a living organism to contribute to normal histogenesis.

### Means of Solving the Problems

The present inventors conducted extensive investigations, found that it is possible to easily determine the presence or absence of the tumorigenic potential in stem cells on the basis of the degree of the dispersion of the cells in fluorescent images obtained by fluorescently labeling the stem cells by introduction of a fluorescence gene or the like, and injecting the stem cells into a fetal mammalian renal primordium, and have developed the present invention.

Accordingly, the present invention relates to the following:

[1] A method of evaluating whether a stem cell is capable of differentiating into a cell that constitutes a desired tissue in a living organism, comprising the following steps of:
(1) transplanting a stem cell to be evaluated into a primordium of a desired tissue of a non-human mammal,
(2) culturing the tissue primordium in vitro, and
(3) determining the possibility that the stem cell differentiates into a cell that constitutes the tissue in a living organism, with the degree of the dispersion of cells derived from the transplanted stem cell in the cultured tissue primordium as an index.
[2] The method described in [1], wherein the stem cell to be evaluated is labeled in a way such that it is distinguishable from the cells of the tissue primordium to which it is transplanted.
[3] The method described in [2], wherein the labeling is fluorescent labeling.
[4] The method described in [1], wherein the tissue is a kidney.
[5] The method described in [2], wherein the stem cell is a mesenchymal stem cell.

### Effect of the Invention

Using the method of the present invention, it is possible to conveniently determine whether a stem cell is a cell that forms a tumor such as a teratoma in a living organism, or a cell capable of differentiating into a wide variety of cells without tumorigenesis to contribute to normal histogenesis in a living organism.

### BRIEF DESCRIPTION OF THE DRAWINGS

FIG. 1 shows time-course changes in a fluorescent image of a metanephron incorporating simian ES cells.

FIG. 2 shows visible light images of a metanephron incorporating simian ES cells. A teratoma formed and differentiation into the three germ layers was noted.

FIG. 3 shows time-course changes in a fluorescent image of a metanephron incorporating swine MSCs.

FIG. 4 shows time-course changes in a fluorescent image of a metanephron incorporating mouse iPS cells.

### MODES FOR EMBODYING THE INVENTION

The present invention provides a method of evaluating whether a stem cell is capable of differentiating into a cell that constitutes a desired tissue in a living organism, comprising the following steps of:

(1) transplanting a stem cell to be evaluated into a primordium of a desired tissue of a non-human mammal,
(2) culturing the tissue primordium in vitro, and
(3) determining the possibility that the stem cell can differentiate into a cell that constitutes the tissue in a living organism, with the degree of the dispersion of cells derived from the transplanted stem cell in the cultured tissue primordium as an index.

Herein, a “stem cell” means an immature cell having the potential for self-replication and the potential for differentiation and proliferation. Stem cells include subpopulations of pluripotent stem cells, multipotent stem cells, unipotent stem cells and the like, depending on their differentiating potential. A pluripotent stem cell means a cell having the potential for differentiating into all tissues and cells that constitute a living organism, although it is unable to become an individual by itself. A multipotent stem cell means a cell having the potential for differentiating into not all kinds of, but a plurality of kinds of, tissues or cells. A unipotent stem cell means a cell having the potential for differentiating into a particular tissue or cell.

Pluripotent stem cells include embryonic stem cells (ES cells), EG cells, iPS cells and the like. ES cells can be produced by culturing an inner cell mass on feeder cells. EG cells can be produced by culturing primordial germ cells in a medium containing mSCF, LIF and bFGF (Cell, 70: 841-847, 1992). iPS cells can be produced by introducing Oct3/4, Sox2 and Klf4 (and also c-Myc or n-Myc as required) into somatic cells (for example, fibroblasts, skin cells and the like) (Cell, 126: p. 663-676, 2006; Nature, 448: p. 313-317, 2007; Nat Biotechnol, 26: p. 101-106, 2008; Cell 131: 861-872, 2007). Stem cells established by culturing an early embryo generated by nuclear-transplanting the nucleus of a somatic cell are also preferable as the pluripotent stem cells (Nature, 385, 810 (1997); Science, 280, 1256 (1998); Nature Biotechnology, 17, 456 (1999); Nature, 394, 369 (1998); Nature Genetics, 22, 127 (1999); Proc. Natl. Acad. Sci. USA, 96, 14984 (1999)), Rideout III et al. (Nature Genetics, 24, 109 (2000)).

Multipotent stem cells include somatic stem cells such as mesenchymal stem cells, hematopoietic stem cells, neural stem cells, bone marrow stem cells, and germ stem cells, and the like. The multipotent stem cell is preferably a mesenchymal stem cell. Mesenchymal stem cells broadly mean a population of stem cells capable of differentiating into all or some of osteoblasts, chondroblasts and lipoblasts and the like, or progenitor cells thereof. Mesenchymal stem cells may have the potential for differentiation into renal erythropoietic-producing cells (Transplantation 85: 1654-1658, 2008). Multipotent stem cells can be isolated from a living organism by a method known per se. For example, mesenchymal stem cells can be collected from bone marrow fluid, peripheral blood, cord blood and the like of a mammal by a publicly known ordinary method. For example, human mesenchymal stem cells can be isolated by culturing and passaging hematopoietic stem cells and the like after bone marrow paracentesis (Journal of Autoimmunity, 30 (2008) 163-171). Multipotent stem cells can also be obtained by culturing the above-described pluripotent stem cells under appropriate induction conditions.

The stem cell to be evaluated is preferably an ES cell, an EG cell, an iPS cell, a multipotent stem cell (for example, mesenchymal stem cell) and the like.

Mammals from which the stem cell used in the present invention is derived include, for example, rodents such as mice, rats, hamsters, and guinea pigs, lagomorphs such as rabbits, ungulates such as swine, bovine, goat, horses, and sheep, carnivores such as dogs and cats, primates such as humans, monkeys, rhesus monkeys, marmosets, and orangutans, chimpanzees, and the like. Since a case has been reported in which such established stem cells exhibited a carcinogenetic potential in large mammals such as dogs and monkeys, although the same was not observed in mice and the like (Xiao-Bing Zhang, et al. JCI 118; 1502,2008), and for other reasons, the method of the present invention is advantageous in evaluating the stem cells of ungulates, carnivores and primates.

The method of the present invention is carried out for the purpose of, for example, determining, prior to performing regenerative therapy for a particular tissue using a stem cell, whether the stem cell is capable of appropriately differentiating into a cell that constitutes the desired tissue. Therefore, the stem cell to be evaluated is a stem cell expected to differentiate into a cell that constitutes a desired tissue (for example, kidney) in a living organism.

“Differentiating into a cell that constitutes a desired tissue in a living organism” means that when a stem cell is transplanted into the desired tissue of or the primordium of the tissue in a living organism, the stem cell differentiates into a cell that constitutes the tissue. For example, regarding pluripotent stem cells and the like, it is known that there are some cases where a stem cell, when transplanted to a tissue of a living organism, fails to differentiate into a cell that constitutes the tissue and produces a teratoma, despite the fact that the stem cell essentially possesses the potential for differentiating into all tissues and cells. Using the method of the present invention, it is possible to conveniently evaluate whether a stem cell, when transplanted into a living organism, is capable of appropriately differentiating into a cell that constitutes a desired tissue, without forming a tumor such as a teratoma as described above.

The choice of “tissue” is not particularly limited; examples include the kidney, brain, spinal cord, stomach, pancreas, liver, thyroid, bone marrow, skin, muscles, lung, gastrointestinal tract (e.g., large intestine, small intestine), blood vessel, heart, thymus, spleen, peripheral blood, testis, ovary, placenta, uterus, bone, skeletal muscle and the like.

The “primordium of a tissue” refers to the site corresponding to the genesis of the tissue in a fetal mammal. For example, the metanephron, which is the renal primordium, can be mentioned. The metanephron is located around the ureteric bud budding site, more specifically between the segment and the lateral plate, in a fetal mammal. The metanephron is preferably the metanephrogenic mesoderm.

The tissue primordium used in the present invention is generally the primordium of a tissue into which the stem cell to be evaluated is expected to differentiate. For example, when the stem cell to be evaluated is expected to differentiate into a cell that constitutes the kidney (for example, erythropoietic-producing cell), the stem cell is transplanted to the renal primordium. The mammal from which the tissue primordium is derived is as described above. The animal species of the stem cell to be evaluated and the animal species of the tissue primordium may be identical or different. For example, to evaluate a human stem cell, the stem cell can be transplanted into the primordium of a tissue of a non-human mammal such as a swine or a rat.

The tissue primordium is separated from a fetal mammal to the outside of a living organism. Since formation of metanephric tissue begins normally at E11.5 in rats and at E9.5 in mice, fetuses at these stages or after are normally used when using the metanephron as the tissue primordium. The starting stage is preferably E14 or after for rats and E12 or after for mice, more preferably E14 to 16 for rats and E12 to 14 for mice. In other mammals, fetuses at similar stages can suitably be used. However, stages therearound are also applicable, provided that conditions are chosen. Separation of the tissue primordium from the fetus can be achieved using a stereoscopic microscope and the like.

Transplantation of stem cells into a tissue primordium is performed using a manipulator, a micropipette and the like under a stereoscopic microscope. The number of cells to be transplanted can be set as appropriate according to the size of the tissue primordium and the like; when using, for example, a rat renal primordium, normally about 1000 to 10000 stem cells are injected. Although the stem cells are normally transplanted into a tissue primordium separated to the outside of the living organism, the stem cells may be transplanted into a tissue primordium in a mammalian fetus, after which the tissue primordium containing the stem cells may be separated from the fetus to the outside of the living organism. For the injection of stem cells into a renal primordium, see Yokoo T, et al. J Am Soc Nephrol 17; 1026,2006 and the like.

The stem cells to be transplanted are preferably isolated and purified ones. “Isolated and purified” means that an operation for removing cells other than the desired stem cells has been performed. The purity of the stem cells is not particularly limited, as far as it can be evaluated by the method of the present invention, and it is normally 10% or more, preferably 50% or more, more preferably 80% or more, most preferably 90% or more (for example, substantially 100%).

It is preferable that the stem cells to be evaluated be labeled in a way such that they are distinguishable from the cells of the tissue primordium to which they are transplanted. Types of labeling include fluorescent labeling, luminescent labeling, radioisotope labeling and the like; because of measurement convenience and the possibility of extensive analysis, fluorescent labeling or luminescent labeling is preferred, with greatest preference given to fluorescent labeling. Labeling of stem cells with fluorescence or luminescence can be achieved by introducing a fluorescence labeling gene or a luminescence labeling gene into the stem cells. Fluorescent or luminescence labeling genes include genes encoding a protein that emits fluorescence or luminescence and genes encoding an enzyme that emits fluorescence or luminescence when mixed with a corresponding fluorescence substrate or luminescence substrate. Examples of the former include genes encoding fluorescent proteins such as GFP, RFP, YFP, CFP, EGFP, and Kusabira-Orange. Examples of the latter include genes encoding enzymes such as luciferase, β-galactosidase, and peroxidase. Examples of the substrate (luminescent) of luciferase include luciferin (and ATP as necessary) and the like. Examples of the substrate (luminescent) of β-galactosidase include a luciferin galactoside substrate (6-O-β-galactopyranosyl-luciferin) and the like. Examples of the substrate of peroxidase include luminol (and hydrogen peroxide as necessary) and the like.

A luminescence or fluorescence labeling gene can be introduced into stem cells by a genetic engineering technique known per se. For example, stem cells are transfected in vitro with a construct (expression vector) wherein the above-mentioned labeling gene is operably connected to the downstream of a promoter operable in the object cell, and the cells are cultivated in a suitable medium, whereby the labeling gene can be introduced in the stem cells.

Examples of the transfection method include biological method, physical method, chemical method and the like. Examples of the biological method include a method using a virus vector, a method utilizing a specific receptor, a cell fusion method (HVJ (Hemagglutinating Virus of Japan), polyethylene glycol (PEG), an electric cell fusion method, and a nuclear fusion method (chromosome transfer)). In addition, examples of the physical method include a microinjection method, an electroporation method, and a method using a gene gun (particle gun). Examples of the chemical method include a calcium phosphate precipitation method, a lipofection method, a DEAE-dextran method, a protoplast method, a red blood cell ghost method, a red blood cell membrane ghost method and a microcapsule method.

Examples of the expression vector include plasmid vector, PAC, BAC, YAC, virus vector, retrovirus vector and the like, and an appropriate one can be selected from these.

The kind of the promoter is not particularly limited as long as it can induce or promote the expression of the labeling gene in the cell incorporating the labeling gene. Examples of the promoter include SRα promoter, CMV promoter, PGK promoter, SV40 promoter, ROSA26 and the like.

The above-mentioned expression vector preferably contains a sequence that terminates transcription of the object mRNA (poly-A, generally called a terminator). In addition, for higher expression of the labeling gene, splicing signal, enhancer region and partial intron of eucaryotic gene can also be connected to the 5′ upstream of a promoter region, between a promoter region and a translational region, or 3′ downstream of a translational region. In addition, the above-mentioned expression vector can further contain a selection marker gene (e.g., drug resistance gene such as neomycin resistance gene, hygromycin resistance gene, ampicillin resistance and the like) to be used for the selection of clone stably harboring the introduced labeling gene.

In addition, a stem cell isolated from a mammal introduced with a luminescence or fluorescence labeling gene may be used. The mammal can be produced by a genetic engineering technique known per se. For example, a luminescence or fluorescence labeling gene is introduced into a germ cell such as fertilized egg, unfertilized egg, spermatozoon and progenitor cell thereof and the like of a mammal by a gene transfer method such as calcium phosphate coprecipitation method, electroporation method, lipofection method, agglutination method, microinjection method, gene gun (particle gun) method, DEAE-dextran method and the like, and an offspring animal derived from the germ cell is obtained, whereby a mammal introduced with a luminescence or fluorescence labeling gene can be produced.

For transgene into a germ cell, use of a construct (expression vector) wherein the object labeling gene is connected to the downstream of a promoter operable in the target mammalian cell is generally advantageous.

Specifically, an expression vector wherein a polynucleotide containing a labeling gene is connected to the downstream of a promoter operable in the target mammalian cell is microinjected into a fertilized egg etc. of the target mammal and the fertilized egg is transplanted into the uterus of a pseudopregnant animal, whereby a transgenic mammal highly expressing a labeling gene can be produced.

Examples of the expression vector include plasmid vector, PAC, BAC, YAC, virus vector, retrovirus vector and the like, and an appropriate one can be selected from these.

The kind of the promoter is not particularly limited as long as the expression of the labeling gene can be induced or promoted in a mammal introduced with the labeling gene. Using a tissue non-specific promoter, a mammal ubiquitously expressing a luminescence or fluorescence labeling gene can be produced. Using a tissue isolated from the mammal, the preservative effect on many kinds of tissues can be simultaneously evaluated by performing a test once. Examples of the tissue non-specific promoter include SRα promoter, CMV promoter, PGK promoter, SV40 promoter, ROSA26, β-actin promoter and the like. Using a tissue specific promoter, moreover, a mammal that specifically expresses a luminescence or fluorescence labeling gene in the object tissue can be produced. For example, a labeling gene can be expressed liver specifically using an α1-AT promoter, skeletal muscle specifically using an β-actin promoter and neuron specifically using an enolase promoter.

The above-mentioned expression vector preferably contains a sequence that terminates transcription of the object mRNA (poly-A, generally called a terminator). In addition, for higher expression of the labeling gene, splicing signal, enhancer region and partial intron of eucaryotic gene can also be connected to the 5′ upstream of a promoter region, between a promoter region and a translational region, or 3′ downstream of a translational region. In addition, the above-mentioned expression vector can further contain a selection marker gene (e.g., drug resistance gene such as neomycin resistance gene, hygromycin resistance gene, ampicillin resistance and the like) to be used for the selection of clone stably harboring the introduced labeling gene.

Next, the tissue primordium containing the stem cells transplanted thereto is cultured in vitro. Cultivation of the tissue primordium can be performed using an ordinary technique of organ culture. For example, cultivation of the tissue primordium can be performed by adding an appropriate medium to a dish, floating a filter thereon, placing the tissue primordium on the filter so that the medium will be supplied to the tissue primordium through the filter, and allowing the dish to stand in an incubator. For this cultivation, culture conditions in common use in tissue culture technology can be used. For example, culturing temperature is normally in the range of about 30-40° C., and preferably exemplified by about 37° C. CO₂ concentration is normally in the range of about 1-10%, and preferably exemplified by about 5%. Humidity is normally in the range of about 70-100%, and preferably exemplified by about 95-100%. Duration of cultivation can be set as appropriate, without being particularly limited, as far as it is enough long to allow an evaluation; when using a rat renal primordium, the duration is normally about 7 to 14 days.

Subsequently, with the degree of the dispersion of cells derived from the transplanted stem cells in the cultured tissue primordium as an index, the possibility that the stem cells can differentiate into cells that constitute the tissue in a living organism is determined. The degree of the dispersion of cells derived from the transplanted stem cells can be determined using a microscope or an appropriate imaging. When the stem cells are labeled with fluorescence or the like, a microscope or appropriate imaging apparatus enabling the detection of the label is used. As shown in Examples below, stem cells capable of differentiating into cells that constitute the tissue in a living organism get dispersed in the tissue primordium as they proliferate, and differentiate into cells that constitute the tissue. When the stem cells are labeled with fluorescence or the like, the dispersion of cells can easily be detected as the dispersion of the label. Meanwhile, stem cells that are unable to differentiate into cells that constitute the tissue in a living organism and form a tumor such as a teratoma do not get dispersed in the tissue primordium, assume the form of a cell mass and form a tumor such as a teratoma. With the proliferation of cells, the size of the cell mass increases. When the stem cells are labeled with fluorescence or the like, a cell mass can easily be detected as the mass of the label. The above-described determination is performed on the basis of such a positive correlation between the degree (or presence or absence) of the dispersion of cells derived from the transplanted stem cells in the tissue primordium and the possibility that the stem cells can differentiate into cells that constitute the tissue in a living organism.

That is, if cells derived from the transplanted stem cells get dispersed as they proliferate in the tissue primordium after cultivation, the stem cells can be judged to be highly likely to be able to differentiate into cells that constitute the tissue in a living organism. Meanwhile, if cells derived from the transplanted stems cell do not get dispersed and exhibit an appearance of a cell mass in the tissue primordium after cultivation, the stem cells can be judged to be highly likely to be unable to differentiate into cells that constitute the tissue in a living organism, and form a tumor such as a teratoma.

Using the method of the present invention, it is possible, for example, to evaluate, prior to conducting regenerative therapy for a particular tissue using a stem cell, whether the stem cell is capable of appropriately differentiating into a cell that constitutes the desired tissue, so that the method is useful in controlling the quality of stem cells. Also, using the method of the present invention, it is easily evaluate the presence or absence of a unwanted cell that has not differentiated well and possess the potential for forming a tumor such as a teratoma when performing regenerative therapy using multipotent stem cells or unipotent stem cells differentiated from pluripotent stem cells in vitro. Furthermore, using the method of the present invention, it is possible to evaluate whether an iPS cell is capable of differentiating into a cell that constitutes a specified tissue or has been reprogrammed into the undifferentiated state and possesses the potential for teratogenesis.

The present invention also provides a kit for evaluating whether a stem cell is capable of differentiating into a cell that constitutes a desired tissue in a living organism. The kit comprises a primordium (for example, metanephron and the like) of a desired tissue separated to the outside of a living organism, used in the method of the present invention. The tissue primordium may be frozen in an appropriate tissue preservation liquid (for example, ET-Kyoto solution and the like).

The kit of the present invention can further comprise a reagent for labeling the stem cell to be evaluated in the method of the present invention in a way such that it is distinguishable from the cells of the tissue primordium to which it is transplanted. The reagent includes a fluorescent labeling reagent, a luminescent labeling reagent, a radioisotope labeling reagent and the like. The reagent is exemplified by an expression vector capable of expressing the above-described fluorescent protein, a combination of an expression vector capable of expressing an enzyme and a fluorescence substrate or luminescence substrate for the enzyme, and the like. The kit of the present invention can still further comprise a reagent for transfecting an expression vector to a stem cell by the above-described method.

The kit of the present invention can further comprise a wide variety of reagents and tools used in the method of the present invention (for example, tools for transplanting stem cells into a tissue primordium (manipulator, micropipette and the like), a dish, filter, or medium for culturing a tissue primordium in vitro, an instruction manual describing the above-described method of the present invention, a microscope and appropriate imaging apparatus enabling the detection of the marker, and the like).

Using the kit of the present invention, it is possible to easily evaluate whether a stem cell is capable of differentiating into a cell that constitutes a desired tissue in a living organism by the method of the present invention.

The present invention is explained in more detail in the following by referring to Examples, which are not to be construed as limitative.

### EXAMPLES

### Example 1

While stem cells, represented by iPS cells, are established by introducing various genes, a case has been reported in which such established stem cells exhibited a carcinogenetic potential in large animals such as dogs and monkeys, although the same was not observed in mice and the like (Xiao-Bing Zhang, et al. JCI 118; 1502,2008). With this in mind, in the present study, simian ES cells labeled with green fluorescent protein (GFP) (Nagata M, et al. J Gene Med 5; 921,2003) and MSCs established from a swine incorporating the red fluorescent protein Kusabira-Orange (Matsunari H, et al. Cloning Stem Cells 10; 313,2008) were used.

It is known that the simian ES cells form a teratoma when transplanted to a living organism, and that the swine MSCs are capable of differentiating into erythropoietin-producing cells that constitute a kidney when transplanted into a renal primordium (Transplantation 85: 1654-1658, 2008).

Injection of stem cells to be tested into a fetal rat renal primordium was performed as reported (Yokoo T, et al. J Am Soc Nephrol 17; 1026,2006). Specifically, 100 to 10000 cells (simian ES cells or swine MSCs) were injected into a fetal rat renal primordium using a mouth pipette under a stereoscopic microscope. After the injection, the renal primordium was subjected to organ culture on a filter-equipped double culture dish by a conventional method for 10 to 14 days. After the cultivation, the renal primordium was examined under a stereoscopic microscope.

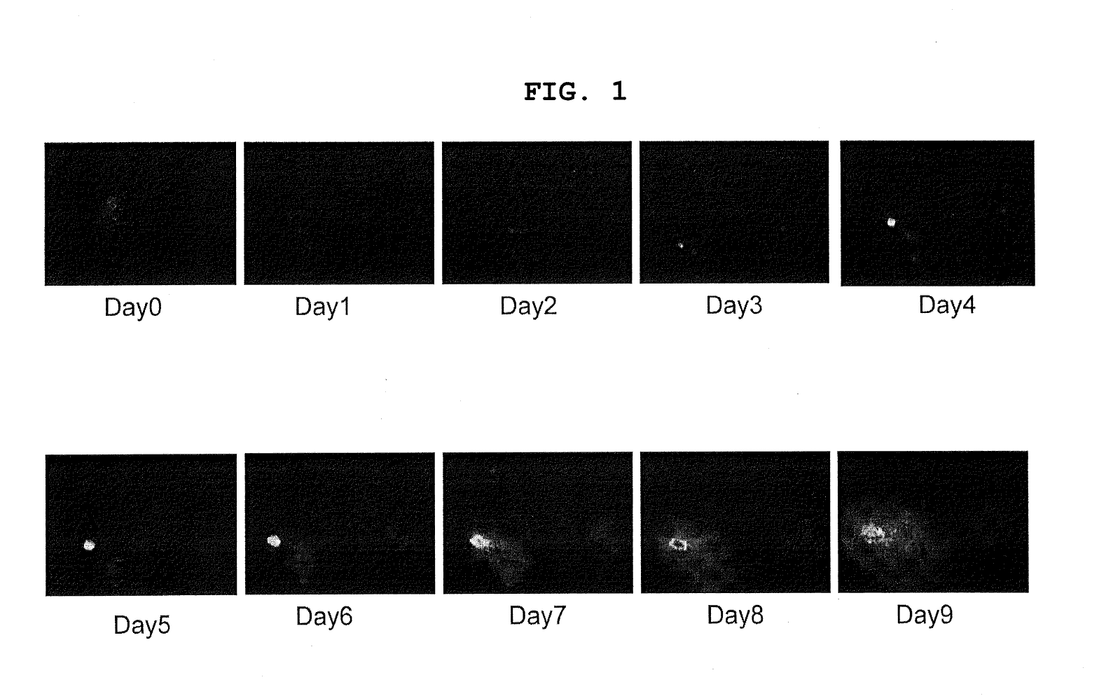

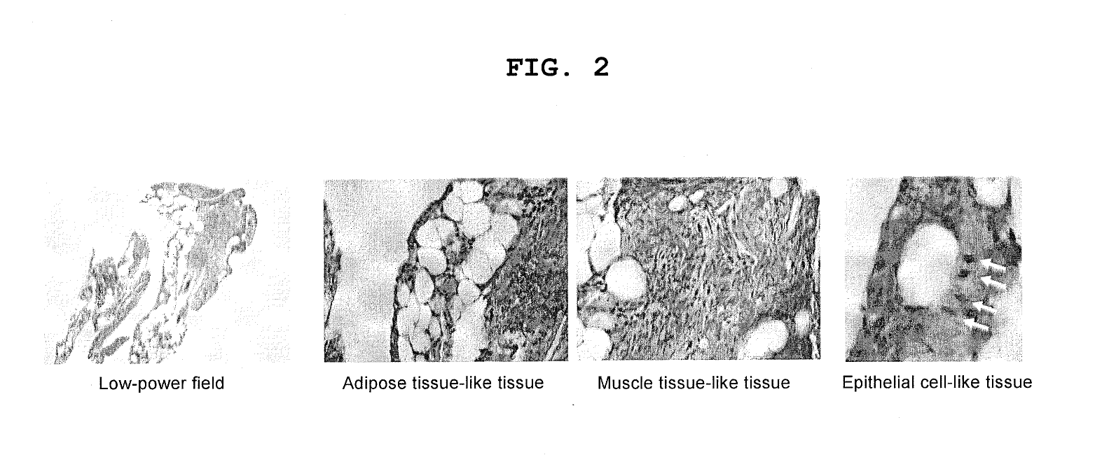

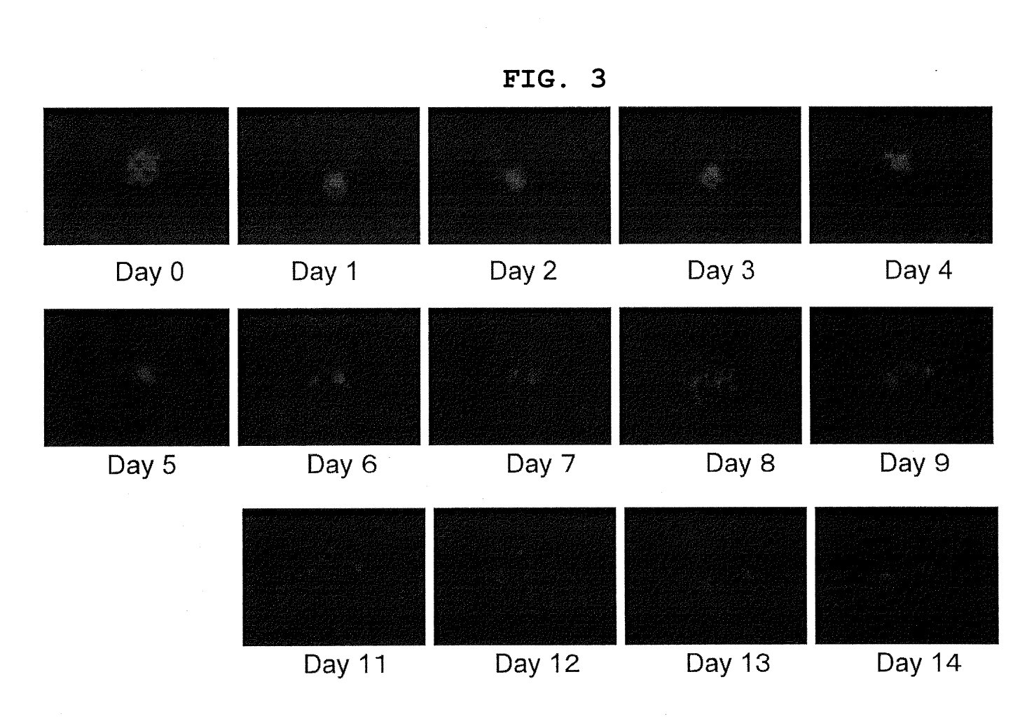

As a result, how the transferred simian ES cells became a single mass and formed a teratoma was revealed by fluorescent images (FIG. 1). Formation of a teratoma was also revealed by light microscopy (FIG. 2). Meanwhile, the swine MSC got dispersed in the renal primordium and differentiated into a kidney without forming a tumor (FIG. 3).

### Example 2

A fetal rat at 15 days of gestation was separated from a pregnant rat; a renal primordium was extirpated from the fetal rat under a stereoscopic microscope and placed on the filter of a filter-equipped double culture dish. Meanwhile, mouse iPS cells established by a reported method (Takahashi K, et al. Nature Protocols 12; 2, 2007) were prepared as a suspension of 1000 to 10000 cells and injected into the rat renal primordium using a mouse pipette under a stereoscopic microscope. After the cell injection, the rat renal primordium was subjected to organ culture by a conventional method for 14 days. After the cultivation, the renal primordium was examined under a stereoscopic microscope.

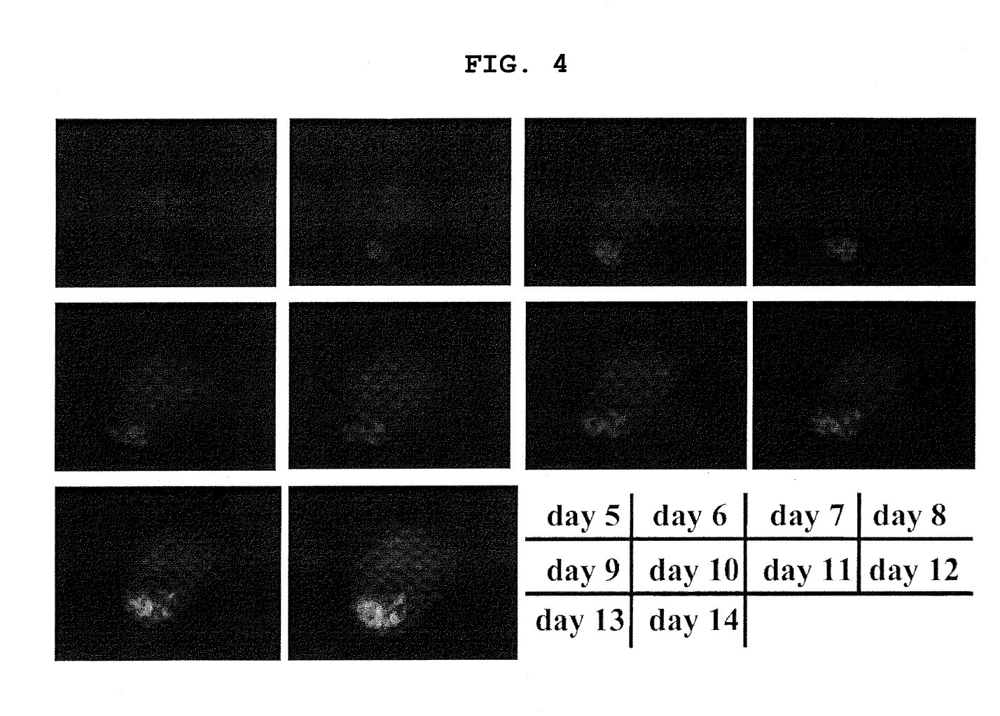

As a result, as with ES cells, how the transferred mouse iPS cells became a single mass and formed a teratoma was revealed by fluorescent images (FIG. 4).

### INDUSTRIAL APPLICABILITY

Using the method of the present invention, it is possible to conveniently determine whether a stem cell is a cell that faults a tumor such as a teratoma in a living organism, or a cell capable of differentiating into a wide variety of cells without tumorigenesis to contribute to normal histogenesis in a living organism.

This application is based on an international patent application PCT/JP2008/073849 (filing date: Dec. 26, 2008), the contents of which are incorporated in full herein.

1. A method of evaluating whether a stem cell is capable of differentiating into a cell that constitutes a desired tissue in a living organism, comprising the following steps of:
(1) transplanting a stem cell to be evaluated into a primordium of a desired tissue of a non-human mammal,
(2) culturing the tissue primordium in vitro, and
(3) determining the possibility that the stem cell differentiates into a cell that constitutes the tissue in a living organism, with the degree of the dispersion of cells derived from the transplanted stem cell in the cultured tissue primordium as an index.

2. The method according to claim 1, wherein the stem cell to be evaluated is labeled in a way such that it is distinguishable from the cells of the tissue primordium to which it is transplanted.

3. The method according to claim 2, wherein the labeling is fluorescent labeling.

4. The method according to claim 1, wherein the tissue is a kidney.

5. The method according to claim 2, wherein the stem cell is a mesenchymal stem cell.
